(19) United States
(12) Plant Patent Application Publication
(10) Pub. No.: US 2011/0010810 P1
(43) Pub. Date: Jan. 13, 2011

(54) GRAPEVINE PLANT NAMED '46 D-2'

(50) ;

(76) Inventor: Mike Gerawan, Reedley, CA (US)

Correspondence Address: MARK D MILLER;KIMBLE, MACMICHAEL & UPTON, 5260 NORTH PALM AVENUE, SUITE 221, FRESNO, CA, 93704, US

(21) Appl. No.: 12/459,932

(22) Filed: Jul. 8, 2009

(51) Int. Cl. A01H 5/00

(52) U.S. Cl. PLT/205

(57) ABSTRACT

A new variety of grapevine plant characterized by producing a medium to large black grape with a very good flavor and good productive capacity that matures for harvesting and shipment in October in the San Joaquin Valley of California. The new variety can be compared to ‘Autumn Royal’ but produces a slightly larger berry with very good flavor.

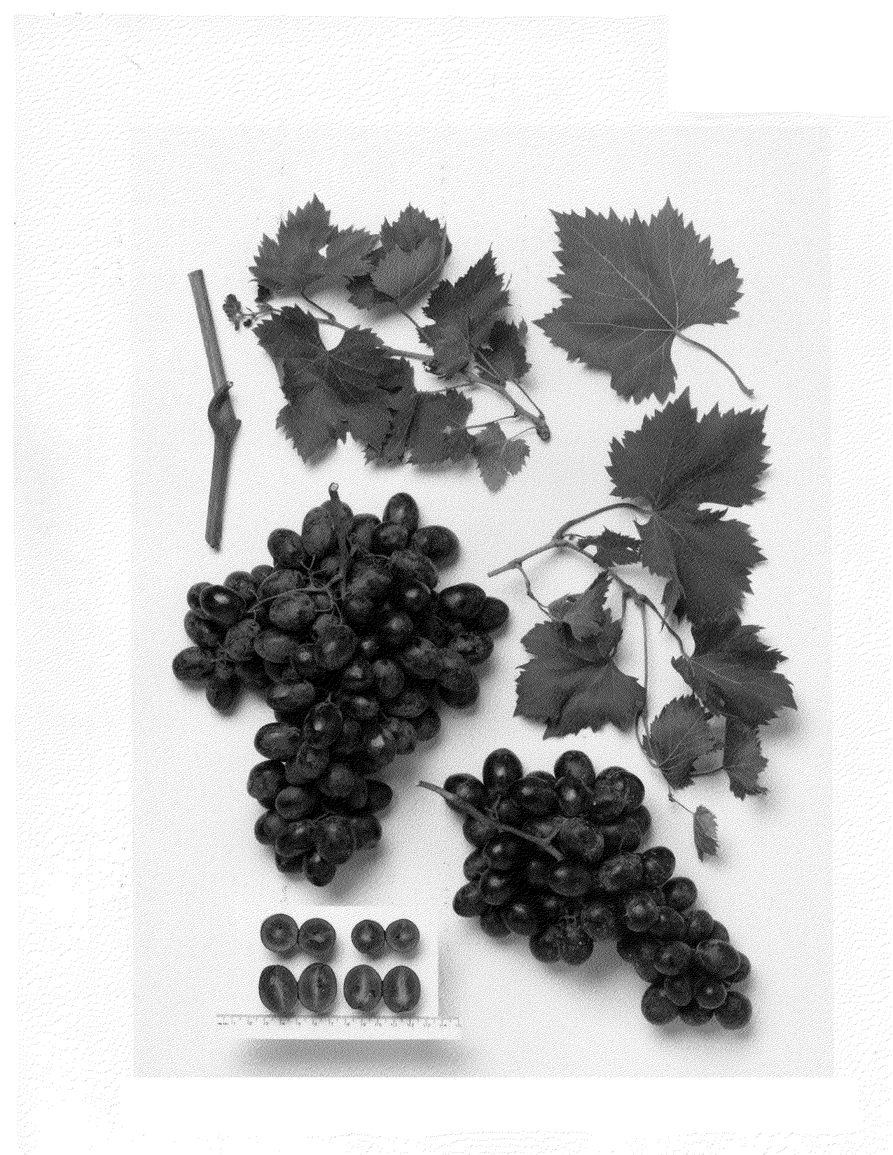

### BACKGROUND OF THE NEW VARIETY

The present invention relates to a new and distinct variety of grapevine plant, Vitis vinifera, and will hereafter be denominated varietally as ‘46 D-2’ and more particularly as a grapevine which produces a medium to large size grape which is mature for harvesting and shipping in October. The new invention resembles ‘Autumn Royal’ (non patented), maturing about one week later.

### ORIGIN AND ASEXUAL REPRODUCTION OF THE NEW VARIETY

The new variety of Vitis vinifera is a naturally occurring branch mutation of ‘Autumn Royal’ (non patented). This bud sport was observed in 2007, maturing one week later with medium to large black berries which are ovate and very sweet.

### SUMMARY OF THE NEW VARIETY

The ‘46 D-2’ grapevine is characterized by producing a medium to large black grape with a very good flavor and good productive capacity that matures for harvesting and shipment in October on the south side of Lincoln Avenue on the west side of the San Joaquin Valley approximately 12 miles west of Fresno. A characteristic distinguishing the new variety and the ‘Autumn Royal’ is that the new variety has a slightly larger berry while having very good flavor.

### BRIEF DESCRIPTION OF THE DRAWINGS

The colored photograph has across the top beginning on the left a piece of a mature branch with young leaves to the right followed by a large mature leaf with the underside displayed. The next row down has a mature cluster of grapes on the left with a leaf cluster to the right displaying both sides of the leaves. Across the bottom four berries are sectioned in half and four longitudinally with a smaller grape cluster to the right.

### DETAILED BOTANICAL DESCRIPTION

Referring more to the horticultural description of the new and distinct variety of grapevine, the following description has been observed in an approximately three year old grapevine under the ecological conditions prevailing at the origin vineyard which is located on the west side of the San Joaquin Valley of central California.

All major color descriptions are by reference to the Dictionary of Color by Maerz & Paul, First Edition, published in 1930. Common colors are also employed.
Vine: This new invention has large vines.
   Height.—42 inches (106.68 cm) higher above ground than ‘Autumn Royal’.
   Vigor.—very good.
   Figure.—with wide cordons forming a ‘T’ shape.
   Regularity of bearing.—regular.
Trunk:
   Size.—medium, 6 inches (15.24 cm) in circumference; 4.5 inches (11.42 cm) above graft.
   Surface texture.—rough, shaggy.
   Color code.—Bark: Kaffa Hamstead Brown+(7-E-9).
Branches:
   Size.—medium, 1.75 inches (4.45 cm) in circumference.
   Cordon.—25 inches (63.5 cm) on one side, 28 inch (71.12 cm) on the other.
   Color.—one year old wood, Pekinese (13-G-10). on the other side, Viridine Gr. (17-I-6).
Canes:
   Color.—Mature cane, Windsor Tan (13-G-12).
   Nodes.—6 nodes on 27.5 inches of cane (69.85 cm).
   Length between nodes.—4.5 inches (11.43 cm).
   Lenticels.—None.
   Shoots.—Length — medium, 3 to 5 inches (7.62-12.7 cm). Shape — semi-droopy Color — Viridine Gr. (17-1-6).
   Tendrils.—Number — two Size — medium, up to 4.53 inches (11.5 cm) Location — at nodes Form — bifurcate Texture — firm Color: CITRINE Rhubarb (14-L-6).
   Buds.—shape — convex Size — width: 0.20 inch (5.0 mm) Length: 0.28 inch (7.1 mm) Fruitfulness — very good Color: Piccadilly Mauvewood+(7-H-10).
Leaves:
   Size.—medium.
   Density.—dense.
   Length.—Average, 5.25 inch (13.34 cm).
   Width.—Average, 3.75 inch (9.53 cm).
   Form.—pentagonal.
   Texture.—upper surface — smooth lower surface — slightly glabrous.
   Color.—upwardly disposed surface — Mt. Vernon Green (23-J-8) downwardly disposed surface — Tarragon (21-B-7) Leaf Vein — Viridine Gr. (17-1-6).
   Apex.—acute.
   Marginal form.—toothed, gently undulate.
   Base.—reverse “U” shape.
   Petioles.—Size: moderate Length: 2.1 inches (5.334 cm) Thickness: 0.08 inch (2.0 mm) Color: Bois de Rose (5-J-9) Sinus: form — upside down U-shape.
   Lobes.—Number: 4 Tooth Size: length: 0.20 inches (5 mm) width: 0.157 inches (4 mm) Tooth Number: 3-5 Tooth Shape: convex.
Inflorescence:
   Size.—large.
   Length.—0.73-0.77 inch (18.5-19.6 cm).
   Shape.—linear.
   Number borne per spur.—at least three per spur.
   Number borne per vine.—many.
Flowers:
   Flowers.—generally heavy.
   Date of bloom.—May 20th.
   Date of full bloom.—May 26th.
   Flower buds.—Shape: Ovate Size: small Diameter: 0.04-0.79 inch (1.0-2.0 mm) Surface texture — glabrous.
   Flower size.—Length: 0.20 inch (5.1 mm).
   Petals.—Shape: Obcordate Color (upper and lower surfaces) — Piccadilly Mauvewood+(7-H-10) Size — small Width: 0.04 inch (1 mm) Length: 0.04-0.79 inch (1-2 mm).
   Sepals.—Apex: acute Shape: triangular Length: 0.04 in (1 mm) Width: 0.79 in (2 mm).
   Pistil.—Color — Flax Pebble-Peanut+3 (12-B-2) Pistil per flower: 1.
   Amount of pollen.—N/A.
   Date of visible berry.—June 1-3 Size — small Diameter: 0.079 inch (2.0 mm).
Fruit:
   Solids.—19.2 brix.
   Acids.—0.31.
   Sugar/acid ratio.—62.01.
   Juice.—Ph — 4.29 Color — Blue Fox (47-E-1) Production — moderate.
   Seeds.—None to 1 vestigial in a few berries.
   Cap stem pedicel.—average Length: 0.58 inch (14.67 mm) Color: Old Bronze+(14-L-7).
   Berries.—Weight: 0.025 pounds (11.34 grams) Size: Medium to Large Form — ovate Number — approximately 60 per bunch Size — average dimension longitudinal axis — 1.18-1.26 inch (30.0-32.0 mm) Size — average dimension in transverse axis — 0.83-0.94 inch (21.0-24.0 mm).
   Clusters.—Size — medium Average length — 8.0 inches (20.32 cm) Average diameter — 6.0 inches (15.24 cm) Weight — 1.5 pounds (681 grams) Compactness — compact Form — conical.
   Stems.—Length — approx. 2.5 inches (6.35 cm) Caliper — 0.24 inch (6.1 mm) Color: GRASS GR (21-L-5).
Ripening: even.
Eating quality: very good.
Use: fresh market.
Keeping qality: very good.
Resistance to disease: unknown.
Harvest time: mid to late October.
Shipping and handling quality: very good.
Skin: Thickness — thin Texture — firm Tendency to Crack — none known Color — Blush Color, Neapolitan Bl+(46-F-12) Ground Color, Romanesque (46-L-10) Lenticels — 0.
Pulp: Color — clear, Slate+Dusk (47-C-6) Flavor — very good Aroma — very mild Texture — firm The above description of a new variety of grapevine is based on the growing conditions on the west side of the San Joaquin Valley of central California. Variations of the usual magnitude such as differences in maturity dates and production may be due to cultural activities such as irrigation, fertilization, pruning, and climatic changes.

1. A new and distinct variety of grapevine plant characterized by a medium to large black grapes that mature for harvest and shipping in mid to late October about seven days later than ‘Autumn Royal’ with very good flavor, the berries being borne on strong woody branches and being well adapted to commercial handling.
